(19) United States
(12) Patent Application Publication
(10) Pub. No.: US 2007/0244074 A1
(43) Pub. Date: Oct. 18, 2007

(54) Stable parenteral formulation of fosphenytoin sodium

(76) Inventors: Sougata Pramanick, Mumbal (IN); Sukhjeet Singh, Mumbal (IN); Suresh Venkataram, Bangalore (IN)

Correspondence Address: BLANK ROME LLP, 600 NEW HAMPSHIRE AVENUE, N.W., WASHINGTON, DC, 20037, US

(21) Appl. No.: 11/405,197

(22) Filed: Apr. 17, 2006

(51) Int. Cl. A61K 31/675

(52) U.S. Cl. 514/94

(57) ABSTRACT

A more stable formulation of 3-(hydroxymethyl)-5,5-diphenylhydantoin disodium phosphate ester, wherein the composition is made more stable by using a solution essentially free of tromethamine.

### TECHNICAL FIELD

Our invention relates to a way to increase the shelf-life stability in parenteral formulations of fosphenytoin sodium, a prodrug of phenytoin and stable composition thereof.

### BACKGROUND OF THE INVENTION

The parenteral formulation of Fosphenytoin sodium is used for the treatment and management of epilepsy and other types of convulsive states.

Fosphenytoin sodium is a prodrug of phenytoin intended as an alternative to parenteral phenytoin sodium. It has no pharmacological activity before its conversion to phenytoin in the body. The reason behind the development of the prodrug is the undesirable properties of the parent drug (phenytoin) formulation. Sodium phenytoin (commercially available under the trade name Dilantitφ) injection used to be sold in a formulation with excessive propylene glycol and a very high pH (12). This causes significant pain at the site of administration, hypotension, progressive limb ischemia distal to the infusion site and other vascular complications including the ‘purple glove’ syndrome. For this reason Dilantin injection is no longer sold although generics are still available. The prodrug Fosphenytoin sodium is available as an aqueous formulation of pH about 9. However many prodrugs have limited shelf lives due to the loss of potency of the compound themselves and generation of hydrolytic degradianits which are either water-soluble or water insoluble and tend to precipitate out where they are insoluble.

Fosphenytoin is a water soluble prodrug of phenytoin, and accordingly its anticonvulsant effects can be attributed to phenytoin. Fosphenytoin is used in hospitals for the short term treatment of epileptic seizures, was first disclosed in U.S. Pat. No. 4,260,769. The pharmacological and toxicological effects of fosphenytoin include those of phenytoin.

The stability of the prodrug is known in the art to be limited by the occurrence of precipitation in the product. This precipitation is related to the degradation of prodrug to phenytoin and the subsequent precipitation of phenytoin out of the solution.

Pharmaceutical compositions of 3-(hydroxymethyl)-5,5-diphenylhydantoin disodium phosphate ester are disclosed in U.S. Pat. No. 4,925,860. This patent acknowledged that the fosphenytoin sodium is prone to degradation. The '860 patent teaches maintaining the pH of 8.3 to 9.4 using suitable organic buffers like tromethamine The '860 patent teaches these buffers are required and essential for stabilizing the formulation and inhibiting the degradation by preventing the pH from dropping. The pH was adjusted by using sodium hydroxide or hydrochloric acid. This pH range is an official requirement as per the US Pharmacopoeia (USP 28, 1995).

U.S. Pat. No. 6,133,248 also describe the use of Fosphenytoin sodium, cyclodextrin and its derivatives and aqueous pharmaceutically acceptable carrier to extend the shelf life of Fosphenytoin sodium.

The patent application WO 9904798 describes a lyophilized form of Fosphenytoin sodium composition where the lyophilized form can be reconstituted by the addition of a pharmaceutically acceptable diluent.

Process of preparation of Fosphenytoin sodium is disclosed in U.S. Pat. No. 4,709,042. Another process for the preparation of Fosphenytoin sodium has been disclosed in the US Patent Application 20050272706.

Numerous references disclose the degradation products of Fosphenytoin sodium of which the following are merely representative examples:
A. Varia et al. “Phenytoin Prodrugs IV: Hydrolysis of Various 3-(Hydroxymethyl) phenytoin Esters”, Journal of Pharmaceutical Sciences 73(8), 1074-1080, 1984.
Manca D, Walker R M et al : Probabilistic approach to the establishment of maximal content limits of impurities in drug formulations: the case of parenteral Diphenylhydantoic acid; Regul Toxicol Pharmacol. Feb.; 29(1):1-14, 1999.
Shinji Narisawa, Valentino J. Stella : Increased shelf-life of fosphenytoin: Solubilization of a degradant, phenytoin, through complexation with (SBE) beta CD ; Journal of Pharmaceutical Sciences, 87(8), 926-930. 2000

The known derivatives of phenytoin include the 5,5-diphenylhydantoins and its salts disclosed in U.S. Pat. No. 4,260,769. These are the prophenytoins for use in the present invention. U.S. Pat. No. 4,260,769 is, therefore, incorporated by reference.

Diphenylhydantoic acid (DPHA) is a degradation product in parenteral formulations of the anticonvulsant phenytoin and the prodrug Fosphenytoin sodium. DPHA has also been reported to be a minor metabolite of phenytoin. U.S. Pat. No. 4,925,860 focuses more attention on the prevention of the acidic hydrolysis of Fosphenytoin sodium to Phenytoin (the acidic pathway). It is shown that pH drop results in increase in the rate of formation of phenytoin coupled with the fact that phenytoin solubility is lowered at lower pH. This decreases the shelf life of the product since phenytoin is water-insoluble and formation of phenytoin in the aqueous formulation results in the saturation of the solution and eventual precipitation of the degradant and the problem of visible particulate matter.

The closest prior art for the present invention is U.S. Pat. No. 4,925,860. We have studied the product produced by the method taught in '860patent and have observed that the product shows a high amount of Phenytoin Related Compound B Diphenylhydantoic acid (DPHA) when stored at 25° & 60% Relative Humidity for 6 months. We have posited that the rise of the impurities might be caused by to alkaline hydrolysis of the prodrug in presence of tromethamine.

Hence, the present invention is aimed to develop tromethamine free parenteral formulations of Fosphenytoin.

### OBJECT

An object of the invention is aimed to provide all aqueous, stable Fosphenytoin Sodium injection composition that is devoid of tromethamine and more stable in comparison with prior art formulations.

One way that we have found to do this is to avoid the alkaline hydrolysis in the aqueous solutions, thereby minimizing impurity formation on the storage conditions.

We have also found that one can reduce the acidic hydrolysis by maintaining/adjusting the required pH of above 8, by using strong acids and bases (hydrochloric acid and sodium hydroxide) rather than weak acids and bases.

### SUMMARY

Our invention provides parenteral compositions of Fosphenytoin which are stable on prolonged storage conditions. We have found that one can improve the stability by formulating fosphenytoin sodium in an aqueous solution, which is devoid of tromethamine, adjusting the pH of the formulation by using strong acid and strong base (sodium hydroxide and hydrochloric acid).

### DETAILED DESCRIPTION OF THE INVENTION

The present invention describes stable parenteral formulations of Fosphenytoin Sodium The pH of our formulation is critical in achieving the stability of the aqueous formulation. At pH lesser than 8, the pathway tends towards acidic hydrolysis of the phospho-ester bond leading to the formation of phenytoin. This may further proceed to form formaldehyde, 5,5-diphenyl-4-imidazolidinone (DIZ), diphenylglycinamide and benzophenone. On the contrary alkaline degradation at higher pH leads to the reversible formation of diphenylhydantoic acid (DPHA—Phenytoin Related Compound B) and an irreversible formation of diphenylglycine (Phenytoin Related Compound A)—as shown in the scheme below:

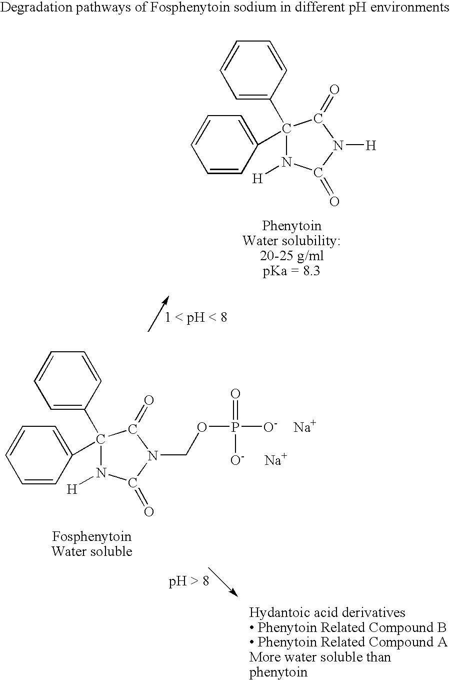

During the development work, the we found that the marketed product Cerebyx® shows a high amount of Phenytoin Related Compound B (DPHA) when stored at 25° & 60% RH for 6 months. The same was established by manufacturing few batches of the qualitative formula similar to the marketed formula and with reduced amount of tromethamine and subsequent stability studies (Example 1 and 2). DPHA has higher solubility than phenytoin and hence is not precipitated. However, we believe that the rise of impurities is attributed to alkaline hydrolysis in presence of tromethamine in the solution.

To support the observation of higher degradation levels in presence of tromethamine, we manufactured several batches without tromethamine, adjusting the pH using strong acid and strong base. We have found that in these batches (Example 3), the amount of the said impurity (DPHA) is significantly lower. On reproducible trials we have found that the formulation is stable in all quality parameters. On storage at 25° C. and 60% RH for 6 months (which is the accelerated storage condition as per the marketed drug's storage instructions), the amount of DPHA was found to be 0.6% or less. This is significantly lesser than the USP impurity limit of 1.5%. The pH of the formulation was seen to remain within the label of the marketed drug (pH 8.6 to 9.0) and well within the USP limits (pH 8.3 to 9.3). However the corresponding study of the marketed formulation and our own formulations with tromethamine results in DPHA content of about 1.5% or more after 6 months. Our data thus shows that in a side-by-side comparison, our tromethamine-free formulation is superior.

After, the extensive stability studies of the marketed formulation and similar formulations prepared in our laboratory, we have established that the presence of a buffer—tromethamine, is responsible for the alkaline hydrolysis of the formulations. This buffer imparts the impurities/degradants (Diphenylhydantoic acid) to the formulation of fosphenytoin when it kept for storage.

Hence we researched whether it would be possible to provide stable parenteral formulations of fosphenytoin which are devoid of buffers. The stability is achieved without using the buffering agent in the formulations, thereby inhibiting the alkaline degradation of Fosphenytoin sodium to Diphenylhydantoic acid, which usually occurs in presence of the buffer.

The present invention further comprises commonly used strong bases and acids such as sodium hydroxide and hydrochloric acid to maintain the pH above 8, whereby avoided acidic hydrolysis of the prodrug used in the formulation.

The following examples, which include preferred embodiments, will serve to illustrate the practice of this invention, it being understood that the particulars shown are by way of example and for purpose of illustrative discussion of preferred embodiments of the invention.

### EXAMPLE-1.

Parenteral formulation of Fosphenytoin sodium similar to marketed composition (A) was prepared as below.

| Ingredients | Qty./mL | |
|---|---|---|
| Fosphenytoin Sodium | 75.00 | mg |
| Tromethamine | 24.22 | mg |

| | |
|---|---|
| Hydrochloric acid | qs to adjust pH |
| Sodium hydroxide | qs to adjust pH |
| Water for Injection | qs to 1 ml |

The buffer tromethamine was dissolved in about 80% water required for the batch. Fosphenytoin sodium was added to this solution and dissolved by stirring. The pH of the solution was adjusted to pH 9.0 using Hydrochloric acid/ Sodium hydroxide and subsequently the volume was made up with Water for Injection The entire operation was carried out with Nitrogen purging and a subsequent blanket of nitrogen during filling into glass vials and sealing them to reduce dissolved oxygen levels.

TABLE 1
Degradation of Fosphenytoin sodium
in presence of Tromethamine.

Composition A

| Storage condition | Assay | DPHA |
|---|---|---|
| Initial | 97.7 | 0.02 |
| 6 months at 25° C. & 60% RH | 94.0 | 1.92 |

### EXAMPLE-2

Parenteral solution of Fosphenytoin sodium with reduced amount of Tromethamine(B) was prepared as below.

| Ingredients | Qty./mL | |
|---|---|---|
| Fosphenytoin Sodium | 75.00 | mg |
| Tromethamine | 12.0 | mg |

| | |
|---|---|
| Hydrochloric acid | qs to adjust pH |
| Sodium hydroxide | qs to adjust pH |
| Water for Injection | qs to 1 ml |

The procedure for preparation adopted was same as in Example-1.

TABLE 2
Degradation of Fosphenytoin sodium in
presence of reduced Tromethamine.

Similar to marketed formulation
with reduced amount of buffer- B

| Storage condition | Assay | DPHA |
|---|---|---|
| Initial | 100.1 | <0.01 |
| 3 months at 25° C. & | 100.0 | 0.53 |
| 60% RH | | |
| 3 months at 25° C. & | 98.6 | 1.52 |
| 75% RH | | |

### EXAMPLE-3

Batches I and II (without Tromethamine)

Parenteral solution of Fosphenytoin sodium without Tromethamine was prepared as below.

| Ingredients | Qty./mL | |
|---|---|---|
| Fosphenytoin Sodium | 75.00 | mg |

| | |
|---|---|
| Hydrochloric acid | qs to adjust pH |
| Sodium hydroxide | qs to adjust pH |
| Water for Injection | qs to 1 mL |

The pH of the Water for injection to be used was adjusted to pH about 11 with Sodium Hydroxide. Fosphenytoin sodium was added to this solution and dissolved by stirring. The pH of the solution was adjusted to pH 9.0 using Hydrochloric acid and subsequently the volume was made up with Water for injection. The entire operation was carried out with nitrogen purging of the solvent and a subsequent blanket of nitrogen was maintained during filling into glass vials and sealing them to reduce dissolved oxygen and oxygen present in free spaces.

TABLE 3
The comparative stability data of the present formulations with
reference to the marketed formulations. (with tromethamine).

| | | | | | Similar to marketed | | | | | |
|---|---|---|---|---|---|---|---|---|---|---|
| | | | Similar to | | formulation with | | Formulation without Tromethamine | | | |
| | Marketed | | marketed | | reduced amount of | | (our invention) | | | |
| Storage | formulation | | formulation- A | | Tromethamine- B | | Batch I | | Batch II | |
| condition | Assay | DPHA | Assay | DPHA | Assay | DPHA | Assay | DPHA | Assay | DPHA |
| Initial | 97.7 | 0.02 | 99.2 | 0.41 | 98.4 | 0.41 | 96.9 | 0.01 | 98.1 | 0.01 |
| 6 months at | 94.0 | 1.92 | 97.0 | 1.48 | 98.5 | 1.57 | 96.3 | 0.60 | 96.8 | 0.46 |
| 25° C. & | | | | | | | | | | |
| 60% RH | | | | | | | | | | |

From the above, it is evident that the presence of tromethamine in the aqueous formulations of Fosphenytoin is prone to alkaline hydrolysis thereby increasing the content of impurities on storage conditions.

1. A stable injectable pharmaceutical composition for the treatment of convulsive states comprising 3-(hydroxymethyl)-5,5-diphenylhydantoin disodium phosphate ester in a therapeutically effective amount, in an aqueous solution having a pH range of from about 8.3 to about 9.3, wherein said aqueous solution is essentially free of any buffer.

2. The invention of claim 1, having less than about 1% of diphenylhydantoic acid after storage for six months at 25° C. and 60% relative humidity.

3. A method for treating convulsive states comprising providing the invention of claim 1 to a patient in need thereof.

4. A method for making a stable injectable pharmaceutical composition comprising 3-(hydroxymethyl)-5,5-diphenylhydantoin disodium phosphate ester, wherein the improvement comprises using an aqueous solution having a pH range of from about 8.3 to about 9.3, said aqueous solution being essentially free from tromethamine.

5. The composition of claim 1, wherein the pH range is achieved by using hydrochloric acid and sodium hydroxide.

6. The composition of claim 1, wherein the concentration of 3-(hydroxymethyl)-5,5-diphenylhydantoin disodium phosphate ester is 75 mg/mL.

7. The composition of claim 1, wherein the composition is free of the buffer tromethamine.
